(19) United States
(12) Patent Application Publication
(10) Pub. No.: US 2025/0270151 A1
(43) Pub. Date: Aug. 28, 2025

(54) FUEL PROCESS AND PLANT

(71) Applicant: Topsoe A/S, Kgs. Lyngby (DK)

(72) Inventors: Sudip DE SARKAR, Kokkedal (DK); Thomas Sandahl CHRISTENSEN, Kgs. Lyngby (DK); Esben Lauge SØRENSEN, Hillerød (DK); David MINGUEZ, København N (DK)

(73) Assignee: Topsoe A/S, Kgs. Lyngby (DK)

(21) Appl. No.: 18/857,492

(22) PCT Filed: Apr. 19, 2023
(86) PCT No.: PCT/EP2023/060138

§ 371 Date: Oct. 17, 2024

(30) Foreign Application Priority Data
Apr. 20, 2022 (DK) PA202200359

(51) Int. Cl. C07C 1/12; B01D 53/54; B01J 8/02; B01J 12/00; B01J 19/24; C01B 3/12; C01B 3/34; C01B 32/40

(52) U.S. Cl. CPC C07C 1/12; B01D 53/54; B01J 8/0278; B01J 12/005; B01J 19/245; C01B 3/12; C01B 3/34; C01B 32/40; B01D 2257/102; B01D 2258/02; B01J 2219/0004; B01J 2219/00132; C01B 2203/0233; C01B 2203/0283; C01B 2203/1241; C01B 2203/146; C01B 2203/148

(57) ABSTRACT

A plant, such as a hydrocarbon plant, or synfuels plant, is provided, with effective use of various streams, in particular carbon dioxide and hydrogen. A method for producing a product stream, such as a hydrocarbon product stream, is also provided. The plant and method of the present invention provide overall better utilization of carbon dioxide and hydrogen, while avoiding build-up of inert components.

### TECHNICAL FIELD

The present invention relates to a plant, such as a hydrocarbon plant, or synfuels plant, with effective use of various streams, in particular carbon dioxide and hydrogen. A method for producing a product stream, such as a hydrocarbon product stream, is also provided. The plant and method of the present invention provide overall better utilization of carbon dioxide and hydrogen, while avoiding build-up of inert components.

### BACKGROUND

Inert components, such as—nitrogen (N₂)—are generally undesired in chemical processes because they reduce partial pressure of the reactants in chemical reactions and increase size of the equipment. The impact becomes even more prominent when processes operate in a loop or where an off-gas from a downstream process is recycled to the front end of the process. Continuous purge is necessary in such situations, to avoid build-up of inert components in the system.

One such process is production of synthetic fuels. Traditional gas-to-liquid (GTL) plants and processes based on Fischer-Tropsch (F-T) synthesis use fossil-based resources as feed, which may contain inert components, such as N₂. Typically, such a plant includes a recycle off-gas from downstream F-T synthesis, whereby inert components can build up in the system. However, a significant part of such off-gas is typically used as a supplemental fuel source in a fired equipment, such as a fired heater. This fuel flow acts as the needed purge of inert components, and is large enough to avoid build-up of inert components in the system.

However, the situation is quite different in a so-called eFuel plant and processes for F-T based synthetic fuel production from non-fossil resources, such as H₂-rich and CO₂-rich feed streams. In such processes and plants, fired equipment may not be necessary. Thus, any inert components in CO₂ rich and/or H₂ rich feeds can build up in the system, unless a significant part of the recycled off-gas from F-T synthesis is purged. Such purging also results in loss of valuable components, derived from expensive H₂ and CO₂ rich feedstock, causing poor overall C-efficiency and H-efficiency of the overall process. Depending on the amount and type of the inert component in the CO₂ and/or H₂ rich feeds, the feasibility of such plant and process can be significantly affected.

There is therefore a need to provide plants and processes for fuel production, in which fired equipment is not included, and where inert build-up can be managed effectively.

### SUMMARY

It is found advantageous to segregate inert components from other constituents of the off-gas before purging. At the same time, recovered valuable components are recycled to the process, increasing overall C-efficiency and H-efficiency.

So, in a first aspect the present invention relates to a plant (X), said plant comprising:
a. a syngas stage (A);
b. a synthesis stage (B); and
c. a nitrogen removal stage (C)
   said plant comprising:
a first feed comprising hydrogen to the syngas stage (A), and a second feed comprising carbon dioxide to the syngas stage (A); or
a combined feed comprising hydrogen and carbon dioxide to the syngas stage (A);
   wherein said syngas stage (A) is arranged to convert at least a portion of said first feed and at least a portion of said second feed—or at least a portion of said combined feed—into a syngas stream, having a higher CO content than the first feed and the second feed, or the combined feed, and feed a syngas stream to the synthesis stage (B);
   wherein said synthesis stage (B) is arranged to receive a syngas stream from the syngas stage (A) and provide at least a product stream and an off-gas stream;
   wherein said nitrogen removal stage (C) is arranged to receive at least a portion of the off-gas stream from the synthesis stage (B) and separate it into a nitrogen-rich purge stream and a purified off-gas stream; and
   wherein said syngas stage (A) is arranged to receive at least a portion of the purified off-gas stream from the nitrogen removal stage (C) and convert it to a syngas stream, preferably in admixture with said first and second feeds or said combined feed.

The plant makes effective use of various streams; in particular CO₂ and H₂. A method for producing a product stream, such as a hydrocarbon product stream is also provided, which uses the plant set out above.

Further details of the technology are provided in the enclosed dependent claims, figures and examples.

### LEGENDS

The technology is illustrated by means of the following schematic illustrations, in which:

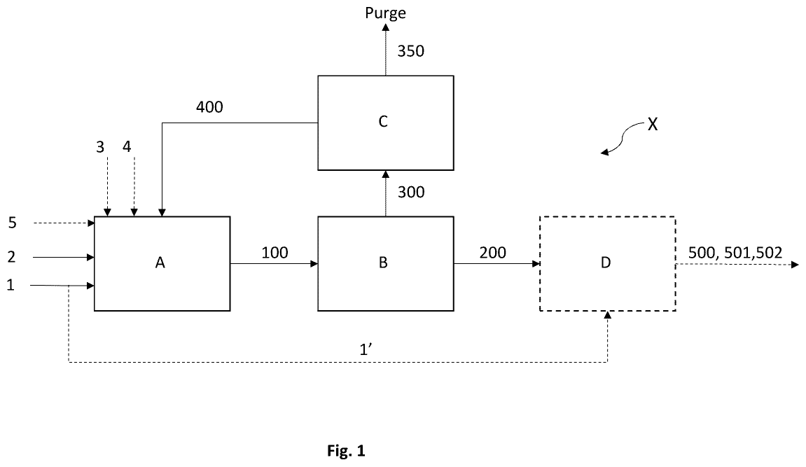

FIG. 1 shows a first layout of the plant of the invention, including nitrogen removal stage (C).

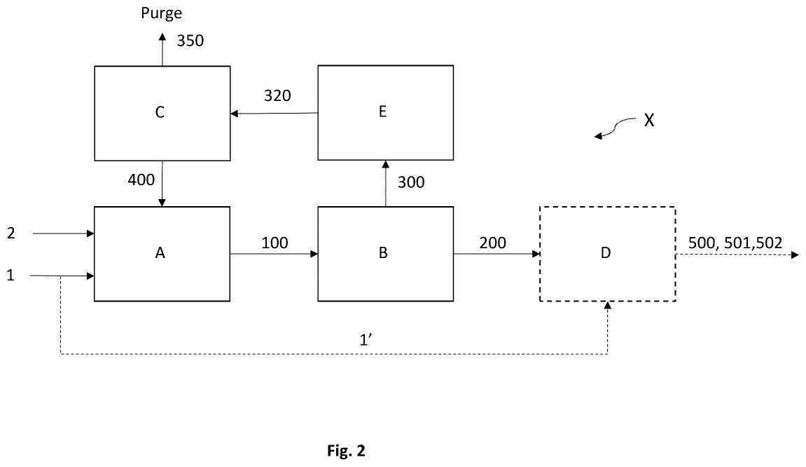

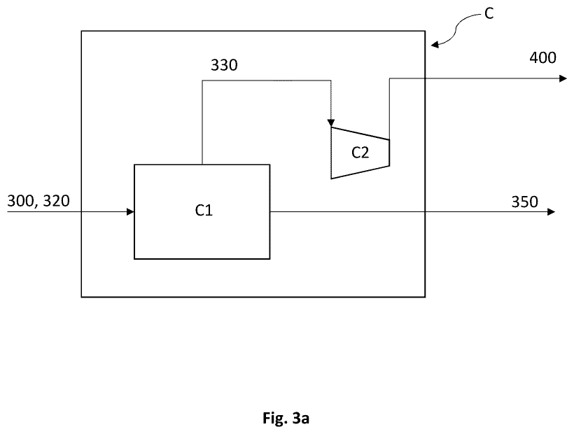

FIG. 2 shows a variation of the plant of FIG. 1, including nitrogen removal stage (C) and off-gas treatment stage (E).

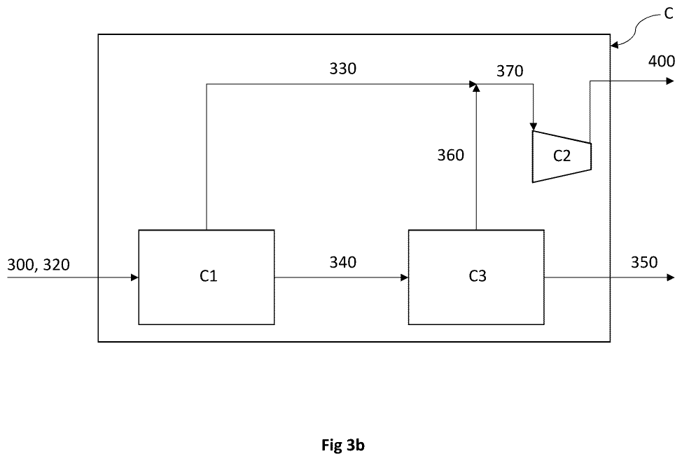

FIG. 3a shows one possible layout of the nitrogen removal stage (C).

FIG. 3b shows another possible layout of the nitrogen removal stage (C).

### DETAILED DISCLOSURE

Unless otherwise specified, any given percentages for gas content are % by volume.

In a first aspect, a plant is provided, said plant comprising:
a. a syngas stage (A);
b. a synthesis stage (B); and
c. a nitrogen removal stage (C)

The plant additionally comprises:
a first feed comprising hydrogen to the syngas stage (A), and a second feed comprising carbon dioxide to the syngas stage (A); or
a combined feed comprising hydrogen and carbon dioxide to the syngas stage (A).

The syngas stage is arranged to convert at least a portion of said first feed and at least a portion of said second feed—or at least a portion of said combined feed—into a syngas stream rich in CO, and feed a syngas stream to the synthesis stage. The term “rich in CO” means the content of CO in this stream is at least 20%, preferably at least 25%. In particular, this syngas stream has a higher CO content than the feeds provided to the syngas stage.

The term “synthesis gas” or “syngas” is meant to denote a gas comprising hydrogen, carbon monoxide and also carbon dioxide and small amounts of other gasses, such as argon, nitrogen, methane, etc. The syngas stream suitably has the following composition (by volume):
40-70% H₂ (dry)
10-40% CO (dry)
2-20% CO₂ (dry)

The syngas stream may additionally contain other components such as methane, steam, and/or nitrogen.

### First Feed

A first feed comprising hydrogen is provided to the syngas stage (A). Suitably, the first feed consists essentially of hydrogen. The first feed of hydrogen is suitably “hydrogen rich” meaning that the major portion of this feed is hydrogen; i.e. over 75%, such as over 85%, preferably over 90%, more preferably over 95%, even more preferably over 99% of this feed is hydrogen. One source of the first feed of hydrogen can be one or more electrolyser units. In addition to hydrogen the first feed may for example comprise steam, nitrogen, argon, carbon monoxide, carbon dioxide, and/or hydrocarbons. In some cases a minor content of oxygen may be present in this feed, typically less than 100 ppm. The first feed suitably comprises only low amounts of hydrocarbon, such as for example less than 5% hydrocarbons or less than 3% hydrocarbons or less than 1% hydrocarbons.

### Second Feed

A second feed comprising carbon dioxide is provided to the syngas stage (A). Suitably, the second feed consists essentially of CO₂. The second feed of CO₂ is suitably “CO₂ rich” meaning that the major portion of this feed is CO₂; i.e. over 75%, such as over 85%, preferably over 90%, more preferably over 95%, even more preferably over 99% of this feed is CO₂. One source of the second feed of carbon dioxide can be one or more exhaust stream(s) from one or more chemical plant(s). One source of the second feed of carbon dioxide can also be carbon dioxide captured from one or more process stream(s) or atmospheric air. Another source of the second feed could be CO₂ captured or recovered from the flue gas for example from fired heaters, steam reformers, and/or power plants.

The second feed may in addition to CO₂ comprise for example steam, oxygen, nitrogen, oxygenates, amines, ammonia, carbon monoxide, and/or hydrocarbons. The second feed suitably comprises only low amounts of hydrocarbon, such as for example less than 5% hydrocarbons or less than 3% hydrocarbons or less than 1% hydrocarbons.

The second feed comprising carbon dioxide may—alternatively or additionally—be a stream comprising CO and CO₂, which is output from an electrolysis section arranged to convert a feed of CO₂ into a stream comprising CO and CO_{2.}

In a particular aspect, a portion of a CO₂ stream is fed directly to the syngas stage (A) as said second feed comprising carbon dioxide, while another portion of this CO₂ stream is fed to an electrolysis section, where it is converted to a stream comprising CO and CO₂. The stream comprising CO and CO₂ may then be fed to the syngas stage (A).

### Combined Feed

As an alternative to separate first feed and second feed, the plant may comprise a combined feed comprising hydrogen and carbon dioxide to the syngas stage. Typically, the hydrogen content of this combined feed is between 40 and 80%, preferably between 50 and 70%.

Typically, the carbon dioxide content of this combined feed is between 15 and 50%, preferably between 20 and 40%. Typically, the carbon monoxide content of this combined feed is between 0 and 10%. Typically, the ratio of hydrogen to carbon dioxide in this combined feed is between 1 and 5, preferably between 2 and 4.

In addition to hydrogen and carbon dioxide, the combined feed may for example comprise steam, nitrogen, argon, carbon monoxide, and/or hydrocarbons. The combined feed suitably comprises only low amounts of hydrocarbon, such as for example less than 5% hydrocarbons or less than 3% hydrocarbons or less than 1% hydrocarbons.

Part of the combined feed may be produced by co-electrolysis of a water/steam feed and a CO₂ feed.

### e-RWGS Section

In one preferred embodiment, the syngas stage (A) comprises an electrically heated reverse water gas shift (e-RWGS) section (I). Electrically-heated reverse water gas shift (e-RWGS) uses an electric resistance-heated reactor to perform a more efficient reverse water gas shift process and substantially reduces or preferably avoids the use of fossil fuels as a heat source.

An e-RWGS section is used in the present invention for carrying out the reverse water-gas shift reaction between CO₂ and H₂. Details of the construction of the e-RWGS section are provided in co-pending application PCT/EP2021/078304.

In one preferred embodiment, the plant comprises:
first feed comprising hydrogen to the e-RWGS section (I), and second feed comprising carbon dioxide to the e-RWGS section (I); or
a combined feed comprising hydrogen and carbon dioxide to the e-RWGS section (I);
   wherein said e-RWGS section (I) is arranged to convert at least a portion of said first feed and at least a portion of said second feed—or at least a portion of said combined feed—into a syngas stream, and feed a syngas stream to the synthesis stage (B); and
   wherein said e-RWGS section (I) is arranged to receive at least a portion of the purified off-gas stream from the nitrogen removal stage (C) and convert it to a syngas stream, preferably in admixture with the first and second feeds or said combined feed.

In an alternative, the syngas stage (A) comprises an methanation section and an autothermal reforming (ATR) section. The plant according to this embodiment comprises:
a first feed comprising hydrogen to the methanation section, and a second feed comprising carbon dioxide to the methanation section; or
a combined feed comprising hydrogen and carbon dioxide to the methanation section;
an oxygen feed to said ATR section.

The methanation section is arranged to convert at least a portion of said first feed and at least a portion of said second feed—or at least a portion of said combined feed—into a methane-containing stream. The ATR section is arranged to receive at least a portion of the methane-containing stream from the methanation section, and said oxygen feed, and convert them to a fourth syngas stream, and the fourth syngas stream is arranged to be fed to said synthesis stage.

In one aspect, the syngas stage (A) comprises a reforming section (II) arranged in parallel to the e-RWGS section (I); wherein said plant comprises a third feed comprising hydrocarbons to said reforming section (II), and wherein said reforming section (II) is arranged to convert at least a portion of said third feed into a second syngas stream, and wherein the first syngas stream from the e-RWGS section (I) is arranged to be combined with said second syngas stream from the reforming section (II) to provide a combined syngas stream, and said combined syngas stream is arranged to be fed to the synthesis stage (B). Details of the third feed comprising hydrocarbons are as below.

In another aspect, the syngas stage (A) comprises a reforming section (II) arranged downstream said e-RWGS section (I); wherein said plant comprises a third feed comprising hydrocarbons to said reforming section (II), and wherein said reforming section (II) is arranged to receive the first syngas stream from the e-RWGS section (I) and provide a second syngas stream, and wherein said second syngas stream is arranged to be fed to the synthesis stage (B).

The reforming section (II) in these aspects is suitably an electrically heated steam methane reforming (e-SMR) section (IIc). Preferably, according to these aspects, the plant (X) does not comprise an oxygen feed to the electrically heated steam methane reforming (e-SMR) section (IIc). The operating temperature of the e-RWGS section (I) is typically 900° C. or more, preferably 1000° C. or more, even more preferably 1100° C. or more.

### ATR Section

The reforming section (II) may be an autothermal reforming (ATR) section (IIa), wherein the plant further comprises a fourth feed comprising steam to the autothermal reforming (ATR) section (IIa).

The ATR reactor typically comprises a burner, a combustion chamber, and a catalyst bed contained within a refractory lined pressure shell. In an ATR reactor, partial combustion of the hydrocarbon containing feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions. More details of ATR and a full description can be found in the art such as “Studies in Surface Science and Catalysis, Vol. 152,” Synthesis gas production for FT synthesis ”; Chapter 4, p.258-352, 2004”. ”.

The oxygen feed to the autothermal reformer may either be oxygen, air, a mixture of air and oxygen, or be an oxidant comprising more than 80% oxygen such as more than 90% oxygen. The oxidant may also comprise other components such as steam, nitrogen, and/or argon. Typically, the oxidant in this case will comprise 5-20% steam.

The “ATR section” may be a partial oxidation “POX” section. A POX section is similar to an ATR section except for the fact that the ATR reactor is replaced by a POX reactor. The POX rector generally comprises a burner and a combustion chamber contained in a refractory lined pressure shell.

The ATR section could also be a catalytic partial oxidation (cPOX) section.

### Third Feed

A third feed comprising hydrocarbons, preferably external to the plant, may be provided to the syngas stage (A). The third feed may additionally comprise other components such as CO₂ and/or CO and/or H₂ and/or steam and/or other components such as nitrogen and/or argon. Suitably, the third feed consists essentially of hydrocarbons or a mixture of hydrocarbons and steam. The third feed of hydrocarbons is suitably “hydrocarbon rich” meaning that the major portion of this feed is hydrocarbons, i.e. over 50%, e.g. over 75%, such as over 85%, preferably over 90%, more preferably over 95%, even more preferably over 99% of this feed is hydrocarbons. The concentration of hydrocarbons in this third feed is determined prior to steam addition (i.e. determined as “dry concentration”).

An example of such third feed can also be a natural gas stream external to the plant. In one aspect, said third feed comprises one or more hydrocarbons selected from methane, ethane, propane or butanes.

The source of the third feed comprising hydrocarbons is suitably external to the plant. The significance of a feed “external to the plant” is that the origin of the feed is not a recycle stream (or a recycle stream further processed or converted) from any synthesis stage in the plant. Possible sources of a third feed comprising hydrocarbons external to the plant include natural gas, LPG, refinery off-gas, naphtha, and renewables, but other options are also conceivable.

Preferably, the ratio of moles of carbon in the third feed comprising hydrocarbons, when external to the plant, to the moles of carbon in CO₂ in the second feed comprising carbon dioxide is less than 0.5.

Suitably, the ratio of moles of carbon in the third feed comprising hydrocarbons, when external to the plant, to the moles of carbon in CO₂ in the second feed is less than 0.3, preferably less than 0.25 and more preferably less than 0.20 or even lower than 0.10. The third feed preferably comprises hydrocarbons is arranged to be fed to the e-RWGS section (I).

Suitably, the third feed comprising hydrocarbons is arranged to be fed to the e-RWGS section (I).

### Synthesis Stage

As noted above, the plant comprises a synthesis stage (B). Suitably, the synthesis stage (B) is arranged to convert said first syngas stream, and optionally said second syngas stream, into at least a product stream and, optionally, a hydrocarbon-containing off-gas stream. It may comprise other process elements, such as—compressor, heat exchanger, separator etc.

The synthesis stage (B) is arranged to receive a syngas stream from the syngas stage (A) and convert it to at least a product stream and an off-gas stream. The content of methane in the synthesis gas stream sent to the synthesis stage (B) is suitably less than 5%, such as less than 3% or even less than 2%.

The synthesis stage (B) may also be arranged to convert the syngas stream into at least a product stream and internal hydrocarbon-containing streams. In this aspect, at least a portion of the internal hydrocarbon-containing streams may be fed to the syngas stage (A) as the third feed comprising hydrocarbons or in addition to the third feed comprising hydrocarbons.

Suitably, the syngas stream at the inlet of said synthesis stage (B) has a hydrogen/carbon monoxide ratio in the range 1.00-4.00; preferably 1.50-3.00, more preferably 1.50-2.10.

In particular, the synthesis stage (B) may be a Fischer-Tropsch (F-T) stage arranged to convert said syngas stream into at least a hydrocarbon product stream and a hydrocarbon-containing off-gas stream in the form of an F-T tail gas stream. In this aspect, at least a portion of said hydrocarbon-containing off-gas stream may be fed to the syngas stage (A) as said third feed comprising hydrocarbons or in addition to said third feed comprising hydrocarbons. This increases the overall carbon efficiency.

In another aspect, the synthesis stage (B) comprises a methanol synthesis stage arranged to convert said syngas stream into at least a hydrocarbon product stream and a hydrocarbon-containing off-gas stream in the form of a methanol product stream.

Additionally, the ratio of H₂:CO₂ provided at the plant inlet may be between 1.0-9.0, preferably 2.5-8.0, more preferably 3.0-7.0, even more preferably 2.8-4.5.

A sixth feed of hydrogen may be arranged to be combined with the first syngas stream, upstream the synthesis stage. This allows the required ratio of H₂:CO₂ to be adjusted as required.

In one embodiment, the plant further comprises an electrolysis section (III) arranged to convert water or steam into at least a hydrogen stream and an oxygen stream, and at least a part of said hydrogen stream from the electrolysis section is arranged to be fed to the syngas stage (A) as said first feed. Additionally, at least a part of the hydrogen stream from the electrolysis section can be comprised as the sixth feed of hydrogen. A part or all of the water or steam, fed to electrolysis section (III), may come from syngas stage (A) or synthesis stage (B).

In the instance where the plant comprises a reforming section (II) being an autothermal reforming (ATR) section (IIa), at least a part of the oxygen stream from the electrolysis section is suitably arranged to be fed to the syngas stage (A) as said fifth feed comprising oxygen.

The electrolysis section (III) may also be arranged to convert a feed of CO₂ into a stream comprising CO and CO₂, wherein at least a part of said stream comprising CO and CO₂ from the electrolysis section (III) is arranged to be fed to the syngas stage (A) as at least a portion of said second feed comprising carbon dioxide.

An electrolysis section may also be arranged upstream the eRWGS to convert a feed of CO₂ and a feed of water or steam into part or all of said combined feed comprising hydrogen and carbon dioxide. In other words, a singly electrolysis section converts both a feed of CO₂ and a feed of water/steam into the combined feed.

In an embodiment, the synthesis gas plant further comprises a gas purification unit and/or a prereforming unit upstream the reforming section. The gas purification unit is e.g. a desulfurization unit, such as a hydrodesulfurization unit. This could also be the case if the hydrocarbon feed is provided to the eRWGS section.

In the prereformer, the hydrocarbon gas will, together with steam, and potentially also hydrogen and/or other components such as carbon dioxide, undergo prereforming according to reaction (iv) in a temperature range of ca. 350-550° C. to convert higher hydrocarbons as an initial step in the process, normally taking place downstream the desulfurization step. This removes the risk of carbon formation from higher hydrocarbons on catalyst in the subsequent process steps. Optionally, carbon dioxide or other components may also be mixed with the gas leaving the prereforming step to form the feed gas.

### Nitrogen Removal Stage (C)

A nitrogen removal stage (C) is arranged to receive at least a portion of the off-gas stream from the synthesis stage (B) and separate it into a nitrogen-rich purge stream and a purified off-gas stream. The purified off-gas stream therefore has a lower nitrogen content than the off-gas stream from the synthesis stage (B).

The nitrogen-rich purge stream suitably has the following gas composition:
25-95% N₂
0-10% CO₂
0-10% CO
0-10% H₂
0-20% CH₄

The purified off-gas stream suitably has the following gas composition:
0-10% N₂
40-70% CO₂
10-30% CO
10-30% H₂
0-20% CH₄

The nitrogen removal stage (C) may comprise any suitable arrangement of sections, such that a sufficient nitrogen removal is achieved. Such sections may be arranged in series or parallel. In particular, sections in the nitrogen removal stage may be one or more combinations of membrane-based units or cryogenic separation stages.

In one aspect, the nitrogen removal stage (C) comprises a first component removal section (C1). This first component removal section (C1) is arranged to receive the off-gas stream and separate it into a third component stream being rich in hydrogen and CO₂, and a fourth component stream being rich in nitrogen and hydrocarbons. preferably wherein at least a portion of said third component stream is arranged to be fed to the syngas stage (A) as a portion of said purified off-gas stream. The section C1 may be (but is not limited to) a membrane-based unit. In this embodiment, the nitrogen removal stage comprises a compression section (C2), in which recovered stream can be compressed before being routed to the syngas stage (A).

The nitrogen removal stage (C) may further comprise a second component removal section (C2), wherein said second component removal section (C2) is arranged to receive the fourth component stream from the first component removal section (C1) and separate it into a fifth component stream being rich in hydrocarbons and a sixth component stream being rich in nitrogen, preferably wherein at least a portion of said fifth component stream is arranged to be fed to the syngas stage (A) as a portion of said purified off-gas stream, optionally in admixture with said third component stream.

Preferably, the nitrogen removal stage (C) comprises a compressor section (C3), said compressor section (C3) being arranged to compress said third component stream, said fifth component stream, or a mixture of said third and said fifth component streams.

Other configurations of sections within the nitrogen removal stage can be designed by the skilled person, as required.

The purified off-gas stream from the nitrogen removal stage (C) is recycled upstream in the plant, allowing for more effective use of this off-gas stream. Therefore, the syngas stage (A) is arranged to receive at least a portion of the purified off-gas stream from the nitrogen removal stage (C) and convert it to a syngas stream. The purified off-gas stream from the nitrogen removal stage (C) is preferably mixed with the first and second feeds or the combined feed prior to being fed to the syngas stage (A).

### Tail Gas Treatment Stage (E)

In one embodiment, the plant further comprises a tail gas treatment stage (E). The tail gas treatment stage (E) is located between the synthesis stage (B) and the nitrogen removal stage (C), and is arranged to treat at least a portion of the tail gas stream from the synthesis stage (B), and provide a treated off-gas stream to the nitrogen removal stage (C).

In one aspect treatment of the tail gas stream from the synthesis stage (B) involves hydrogenation. Therefore, the tail gas treatment stage (E) may comprise a hydrogenation section, arranged to hydrogenate at least a portion of the tail gas stream from the synthesis stage (B). The purpose of the hydrogenation section is conversion of unsaturated hydrocarbons, present in off-gas, to saturated hydrocarbons.

Additionally, the off-gas treatment stage (E) may comprise a water gas shift (WGS) section after a hydrogenation section. The WGS section may comprise at least one WGS unit and if WGS section is present, steam is additionally fed to WGS section. The purpose of the WGS section is conversion of carbon monoxide in the off-gas stream to carbon dioxide. The off-gas treatment stage (E) may further comprise a pre-conversion section after the WGS section. The purpose of the pre-conversion section is conversion of all hydrocarbons in the off-gas to hydrogen, carbon monoxide, carbon dioxide and steam.

In a preferred embodiment, the off-gas treatment stage (E) comprises a reforming section, such as a steam methane reforming (SMR) section, downstream the pre-conversion section. However, presence of higher hydrocarbons (HHC) could be a problem for SMR, so a preconversion step is used. This concept is known as tail gas reforming. The most preferred embodiment of the off-gas treatment stage (E) is thus—in order—hydrogenation section, WGS section, pre-conversation section and SMR section.

Steam methane reforming further increases the H₂ and CO₂ content of the off-gas stream, facilitating the separation.

In all cases, the treated off-gas stream should be allowed to cool before entering the nitrogen removal stage (C).

### Additional Aspects

The product stream from the syngas stage (A) may be subjected to one or more upgrading steps, so that the required product grade can be achieved. Therefore, the plant suitably comprises a Product Work Up (PWU) unit (D) arranged to convert the product stream into one or more purified product streams.

A sixth feed of hydrogen may be arranged to be combined with the syngas stream, upstream the synthesis stage and/or be arranged to be fed to the PWU unit. Suitably, at least a part of said hydrogen stream from the electrolysis section is comprised as said sixth feed of hydrogen.

### Method

As above, a method for producing a product stream, such as a hydrocarbon stream, is provided. All details of the plant set out above are relevant to the method of the invention, mutatis mutandis.

A plant (X) is provided, as defined herein. First feed comprising hydrogen, and second feed comprising carbon dioxide, or the combined feed comprising hydrogen and carbon dioxide are supplied to the syngas stage (A). If required, at least a part of a third feed comprising hydrocarbons is supplied to the syngas stage (A).

At least a portion of said first feed and at least a portion of said second feed—or at least a portion of said combined feed—is converted into a syngas stream, in the syngas stage (A). The syngas stream from the syngas stage (A) is fed to the synthesis stage (B), and a product stream and an off-gas stream are produced from said syngas stream in said synthesis stage (B).

At least a portion of the off-gas stream is supplied from the synthesis stage (B) to said nitrogen removal stage (C), where it is separated it into a nitrogen-rich purge stream and a purified off-gas stream. At least a portion of the purified off-gas stream from the nitrogen removal stage (C) is fed to the syngas stage (A) and converted to a syngas stream, preferably in admixture with said first and second feeds or said combined feed.

In one preferred aspect of the method, the syngas stage (A) comprises an electrically heated reverse water gas shift (e-RWGS) section (I), and the plant comprises:
first feed comprising hydrogen to the e-RWGS section (I), and second feed comprising carbon dioxide to the e-RWGS section (I); or
a combined feed comprising hydrogen and carbon dioxide to the e-RWGS section (I).

According to this aspect, the method comprises a first step of: converting at least a portion of said first feed and at least a portion of said second feed—or at least a portion of said combined feed—into a syngas stream in the e-RWGS section (I). A syngas stream is provided to the synthesis stage (B).

At least a portion of the purified off-gas stream is supplied from the nitrogen removal stage (C) to the e-RWGS section (I) and converted to a syngas stream, preferably in admixture with said first and second feeds or said combined feed.

In the case where the synthesis stage (B) comprises a Fischer-Tropsch (F-T) unit, the method may comprise converting the syngas stream into at least a hydrocarbon product stream and a hydrocarbon-containing off-gas stream in the form of an F-T tail gas stream in the F-T unit.

Other aspects and advantages of the method, and embodiments thereof, correspond to the advantages of the plant, and embodiments thereof, and therefore need not be described in further detail here.

### Specific Embodiments

FIG. 1 shows a first layout of the plant used in the process of the invention. The plant (X) comprises a syngas stage (A), a synthesis stage (B), nitrogen removal stage (C) and product work-up stage (D). The first feed (1), comprising hydrogen, and the second feed (2), comprising carbon dioxide, are supplied to syngas stage (A) which produces syngas stream (100) after separation of process condensate (not shown). This syngas stream (100) has a higher CO content than the first feed (1) and the second feed (2), or the combined feed (8) inputted to the syngas stage (A). Second feed (2) and/or first feed (1) comprise some amounts of nitrogen, which acts as inert. The syngas stream (100) from syngas stage (A) is then fed to synthesis stage (B), where it is converted to at least one product hydrocarbon stream (200) and an off-gas stream (300), having a higher nitrogen concentration than that of the feed streams. Then, at least a part of the off-gas (300) is sent to nitrogen removal stage (C) where purge stream (350) rich in nitrogen is purged out and a nitrogen lean purified off-gas stream (400) is routed back to syngas stage (A). The hydrocarbon stream (200) is (optionally) further routed to product work-up stage (D) for final product(s) (500, 501, 502). A part of the first feed (1′) can be sent to product work-up stage (D).

The syngas stage (A) may comprise an electrically heated reverse water gas shift (eRWGS) section. An eRWGS section may comprise multiple eRWGS units. Alternatively, it may comprise a methanation section and autothermal reforming section. A methanation section may comprises multiple methanation units, arranged in various conceivable layouts. In case the syngas stage (A) comprises autothermal section, a third feed (3), comprising oxygen, feed is required.

FIG. 2 shows a layout similar to FIG. 1, additionally comprising an off-gas treatment stage (E) between synthesis stage (B) and nitrogen removal stage (C). At least a part of the off-gas (300) is passed through off-gas treatment stage (E). In one embodiment, off-gas treatment stage may comprise a hydrogenation section, comprising at least one hydrogenation unit. The purpose of the hydrogenation section is conversion of unsaturated hydrocarbons, present in off-gas, to saturated hydrocarbons.

Additionally, the off-gas treatment stage (E) may comprise a water gas shift (WGS) section after a hydrogenation section. The WGS section may comprise at least one WGS unit and if WGS section is present, steam is additionally fed to WGS section. The purpose of the WGS section would be conversion of carbon monoxide in the off-gas stream to carbon dioxide. The off-gas treatment stage (E) may further comprise a pre-conversion section after WGS section. The purpose of the pre-conversion section is conversion of all hydrocarbons in off-gas to hydrogen, carbon monoxide, carbon dioxide and steam. The treated off-gas stream (320) is then sent to nitrogen removal stage (C) where purge stream (350) rich in nitrogen is taken out and a nitrogen lean purified off-gas stream (400) is routed back to syngas stage (A).

Off-gas treatment ensures conversion of most of the carbon monoxide, hydrocarbons present in off-gas to hydrogen and carbon dioxide. This would make it possible to use one separation section in stage (C) with reduced loss of carbon and hydrogen through purge stream.

FIG. 3a shows one possible embodiment of nitrogen removal stage (C). In this embodiment, at least a part of off-gas (300) or treated off-gas (320) from synthesis stage (B) is sent to first component removal section (C1). The section C1, can comprise (but not limited to) a membrane-based unit to produce first nitrogen depleted stream (330) and a purge stream (350) enriched in nitrogen. In one embodiment, recovered stream (330) can be compressed in a compression section (C2) before routing the purified off-gas stream (400) to syngas stage (A).

FIG. 3b shows another embodiment of nitrogen removal stage (C). In this embodiment, at least a part of off-gas (300) or treated off-gas (320) from synthesis stage (B) is sent to first component removal section (C1). The section C1, can comprise (but not limited to) a membrane-based unit to produce first nitrogen depleted stream (330). The remaining stream from C1, richer in nitrogen, is then sent to second component removal section (C3), which may comprise at least one membrane-based unit and/or a cryogenic unit to separate most of the nitrogen. The separated nitrogen rich stream (350) is purged out from the system. The second nitrogen depleted stream (360), from section C3, can be mixed with purified stream from (330) section C1 to make a mixed purified stream (370). In one embodiment, the mixed purified stream (370) can further be compressed in a compression section (C2) before routing the purified off-gas stream (400) to syngas stage (A).

The following list of numbered aspects is provided:

Aspect 1. A plant (X), said plant comprising:
a. a syngas stage (A);
b. a synthesis stage (B); and
c. a nitrogen removal stage (C)
   said plant comprising:
a first feed (1) comprising hydrogen to the syngas stage (A), and a second feed (2) comprising carbon dioxide to the syngas stage (A); or
a combined feed (8) comprising hydrogen and carbon dioxide to the syngas stage (A);
   wherein said syngas stage (A) is arranged to convert at least a portion of said first feed (1) and at least a portion of said second feed (2)—or at least a portion of said combined feed (8)—into a syngas stream (100) having a higher CO content than the first feed (1) and the second feed (2), or the combined feed (8), and feed a syngas stream (100) to the synthesis stage (B);
   wherein said synthesis stage (B) is arranged to receive a syngas stream (100) from the syngas stage (A) and provide at least a product stream (200) and an off-gas stream (300);
   wherein said nitrogen removal stage (C) is arranged to receive at least a portion of the off-gas stream (300) from the synthesis stage (B) and separate it into a nitrogen-rich purge stream (350) and a purified off-gas stream (400); and
   wherein said syngas stage (A) is arranged to receive at least a portion of the purified off-gas stream (400) from the nitrogen removal stage (C) and convert it to a syngas stream, preferably in admixture with said first and second feeds (1, 2) or said combined feed (8).

Aspect 2. The plant (X) according to aspect 1, wherein said syngas stage (A) comprises an electrically heated reverse water gas shift (e-RWGS) section (I), wherein said plant comprises:
first feed (1) comprising hydrogen to the e-RWGS section (I), and second feed (2) comprising carbon dioxide to the e-RWGS section (I); or
a combined feed (8) comprising hydrogen and carbon dioxide to the e-RWGS section (I);
   wherein said e-RWGS section (I) is arranged to convert at least a portion of said first feed (1) and at least a portion of said second feed (2)—or at least a portion of said combined feed (8)—into a syngas stream (100), and feed a syngas stream (100) to the synthesis stage (B); and
   wherein said e-RWGS section (I) is arranged to receive at least a portion of the purified off-gas stream (400) from the nitrogen removal stage (C) and convert it to a syngas stream, preferably in admixture with the first and second feeds (1, 2) or said combined feed (8).

Aspect 3. The plant (X) according to aspect 1, wherein said syngas stage (A) comprises an methanation section and an autothermal reforming (ATR) section; wherein said plant comprises:
a first feed (1) comprising hydrogen to the methanation section, and a second feed (2) comprising carbon dioxide to the methanation section; or
a combined feed (8) comprising hydrogen and carbon dioxide to the methanation section;
an oxygen feed (5) to said ATR section;
   wherein said methanation section is arranged to convert at least a portion of said first feed (1) and at least a portion of said second feed (2)—or at least a portion of said combined feed (8)—into a methane-containing stream, and;
   wherein said ATR section is arranged to receive at least a portion of the methane-containing stream from the methanation section, and said oxygen feed (5), and convert them to a fourth syngas stream, and wherein said fourth syngas stream is arranged to be fed to said synthesis stage (B).

Aspect 4. The plant according to any one of the preceding aspects, wherein said plant (X) additionally comprises a third feed (3) comprising hydrocarbons to the syngas stage (A), preferably wherein the ratio of moles of carbon in the third feed (3) comprising hydrocarbons, when external to the plant, to the moles of carbon in CO₂ in the second feed (2) comprising carbon dioxide is less than 0.5.

Aspect 5. The plant according to aspect 4, wherein the ratio of moles of carbon in the third feed (3) comprising hydrocarbons, when external to the plant, to the moles of carbon in CO₂ in the second feed (2) is less than 0.3, preferably less than 0.25 and more preferably less than 0.20 or even lower than 0.10.

Aspect 6. The plant according to any one of aspects 4-5, wherein said third feed (3) comprising hydrocarbons is arranged to be fed to the e-RWGS section (I).

Aspect 7. The plant according to any one of the preceding aspects, wherein said syngas stage (A) comprises a reforming section (II) arranged in parallel to said e-RWGS section (I); wherein said plant comprises a third feed (3) comprising hydrocarbons to said reforming section (II), and wherein said reforming section (II) is arranged to convert at least a portion of said third feed (3) into a second syngas stream (40), and wherein the first syngas stream (20) from the e-RWGS section (I) is arranged to be combined with said second syngas stream (40) from the reforming section (II) to provide a combined syngas stream (100), and said combined syngas stream (100) is arranged to be fed to the synthesis stage (B).

Aspect 8. The plant according to any one of aspects 1-6, wherein said syngas stage (A) comprises a reforming section (II) arranged downstream said e-RWGS section (I); wherein said plant comprises a third feed (3) comprising hydrocarbons to said reforming section (II), and wherein said reforming section (II) is arranged to receive the first syngas stream (20) from the e-RWGS section (I) and provide a second syngas stream (40), and wherein said second syngas stream (40) is arranged to be fed to the synthesis stage (B).

Aspect 9. The plant according to any one of the preceding aspects, wherein the content of methane in the synthesis gas stream (100) sent to the synthesis stage (B) is less than 5%, such as less than 3% or even less than 2%.

Aspect 10. The plant according to any one of aspects 7-9, wherein the reforming section (II) is an electrically heated steam methane reforming (e-SMR) section (IIc).

Aspect 11. The plant according to any one of aspects 7-10, wherein the reforming section (II) is an autothermal reforming (ATR) section (IIa), and wherein the plant (X) further comprises a fourth feed (4) comprising steam to the autothermal reforming (ATR) section (IIa).

Aspect 12. The plant according to any one of aspects 7-11, wherein the reforming section (II) is an electrically heated steam methane reforming (e-SMR) section (IIc), and wherein the plant (X) does not comprise an oxygen feed (5) to the electrically heated steam methane reforming (e-SMR) section (IIc).

Aspect 13. The plant according to any one of aspects 7-12, wherein at least a portion of said second feed (2) comprising carbon dioxide is fed to the reforming section (II).

Aspect 14. The plant according to any one of the preceding aspects, further comprising a tail gas treatment stage (E) located between the synthesis stage (B) and the nitrogen removal stage (C), said tail gas treatment stage (E) being arranged to treat at least a portion of the tail gas stream (300) from the synthesis stage (B), and provide a treated off-gas stream (320) to the nitrogen removal stage (C).

Aspect 15. The plant according to aspect 14, wherein the tail gas treatment stage (E) comprises a hydrogenation section, arranged to hydrogenate at least a portion of the tail gas stream (300) from the synthesis stage (B), and optionally wherein the tail gas treatment stage (E) further comprises a water gas shift (WGS) section and a steam feed, arranged downstream said hydrogenation section, further optionally wherein the tail gas treatment stage (E) further comprises a pre-conversion section arranged downstream the water gas shift (WGS) section.

Aspect 16. The plant according to any one of aspects 14-15, wherein the tail gas treatment stage (E) comprises—in order—a hydrogenation section, a WGS section, a pre-conversation section and an SMR section.

Aspect 17. The plant according to any one of the preceding aspects, wherein the nitrogen removal stage (C) comprises a first component removal section (C1), wherein said first component removal section (C1) is arranged to receive the off-gas stream (300, 320) and separate it into a third component stream (330) being rich in hydrogen and CO₂, and a fourth component stream (340) being rich in nitrogen and hydrocarbons, preferably wherein at least a portion of said third component stream (330) is arranged to be fed to the syngas stage (A) as a portion of said purified off-gas stream (400).

Aspect 18. The plant according to aspect 17, wherein the nitrogen removal stage (C) further comprises a second component removal section (C3), wherein said second component removal section (C3) is arranged to receive the fourth component stream (340) from the first component removal section (C1) and separate it into a fifth component stream (360) being rich in hydrocarbons and a sixth component stream (350) being rich in nitrogen, preferably wherein at least a portion of said fifth component stream (360) is arranged to be fed to the syngas stage (A) as a portion of said purified off-gas stream (400), optionally in admixture with said third component stream (330).

Aspect 19. The plant according to any one of aspects 17-18, wherein the nitrogen removal stage (C) comprises a compressor section (C2), said compressor section (C2) being arranged to compress said third component stream (330), said fifth component stream (360), or a mixture (370) of said third and said fifth component streams.

Aspect 20. The plant according to any one of the preceding aspects, wherein the operating temperature of the e-RWGS section (I) is 900° C. or more, preferably 1000° C. or more, even more preferably 1100° C. or more.

Aspect 21. The plant according to any one of the preceding aspects, wherein the syngas stream (100) at the inlet of said synthesis stage (B) has a hydrogen/carbon monoxide ratio in the range 1.00-4.00; preferably 1.50-3.00, more preferably 1.50-2.10.

Aspect 22. The plant according to any one of the preceding aspects, wherein the ratio of H₂:CO₂ provided at the plant inlet is between 1.0-9.0, preferably 2.5-8.0, more preferably 3.0-7.0, even more preferably 2.8-4.5.

Aspect 23. The plant according to aspect 22, wherein the synthesis stage (B) is an FT synthesis stage and the H₂:CO₂-ratio provided at the plant inlet is preferably in the range of 3.0-7.0 or more preferably from 3.0-6.0 and most preferably 2.8-4.50.

Aspect 24. The plant according to any one of aspects 4-23, wherein said third feed (3) comprising hydrocarbons is a natural gas feed.

Aspect 25. The plant according to any one of the preceding aspects, wherein the synthesis stage (B) is arranged to convert said syngas stream (100) into at least a product stream (200) and internal hydrocarbon-containing streams.

Aspect 26. The plant according to aspect 25, wherein at least a portion of said internal hydrocarbon-containing streams are fed to the syngas stage (A) as said third feed (3) comprising hydrocarbons or in addition to said third feed (3) comprising external hydrocarbons.

Aspect 27. The plant according to any one of the preceding aspects, wherein the synthesis stage (B) comprises a Fischer-Tropsch (F-T) unit arranged to convert said syngas stream (100) into at least a raw hydrocarbon product stream and a hydrocarbon-containing off-gas stream in the form of an F-T tail gas stream.

Aspect 28. The plant according to any one of aspects 1-26, wherein the synthesis stage (B) is a methanol synthesis stage arranged to convert said syngas stream into at least a hydrocarbon product stream and a hydrocarbon-containing off-gas stream in the form of a methanol product stream.

Aspect 29. The plant according to any one of the preceding aspects, wherein the plant comprises a Product Work UP (PWU) unit (D) arranged to convert said product stream (200) into one or more purified product streams (500, 501, 502).

Aspect 30. The plant according to any one of the preceding aspects, further comprising an electrolysis section (III) arranged to convert water or steam into at least a hydrogen stream and an oxygen stream (11), and wherein at least a part of said hydrogen stream from the electrolysis section is arranged to be fed to the syngas stage (A) as at least a portion of said first feed (1).

Aspect 31. The plant according to any one of the preceding aspects, comprising a sixth feed of hydrogen arranged to be combined with the syngas stream, upstream the synthesis stage and/or arranged to be fed to the PWU unit (D).

Aspect 32. The plant according to any one of aspects 30-31, wherein at least a part of said hydrogen stream from the electrolysis section is comprised as said sixth feed of hydrogen.

Aspect 33. The plant according to any one of aspects 30-32, wherein the plant comprises a reforming section (II) being an autothermal reforming (ATR) section (IIa), and wherein at least a part of the oxygen stream from the electrolysis section is arranged to be fed to the syngas stage (A) as said oxygen feed (5).

Aspect 34. The plant according to any one of aspects 30-33, wherein the electrolysis section (III) is further arranged to convert a feed of CO₂ into a stream comprising CO and CO₂, and wherein at least a part of said stream comprising CO and CO₂ from the electrolysis section (III) is arranged to be fed to the syngas stage (A) as at least a portion of said second feed (2) comprising carbon dioxide.

Aspect 35. The plant according to any one of the preceding aspects, wherein an electrolysis section is arranged to convert a feed of CO₂ and a feed of water or steam into said combined feed (8) comprising hydrogen and carbon dioxide.

Aspect 36. A method for producing a product stream, such as a hydrocarbon stream, said method comprising the steps of:
providing a plant (X) as defined in any one of the preceding aspects;
supplying first feed (1) comprising hydrogen to the syngas stage (A), and a second feed (2) comprising carbon dioxide to the syngas stage (A); or
supplying a combined feed (8) comprising hydrogen and carbon dioxide to the syngas stage (A);
optionally, supplying at least a part of a third feed (3) comprising hydrocarbons, to the syngas stage (A);
converting at least a portion of said first feed (1) and at least a portion of said second (2) feed—or at least a portion of said combined feed (8)—into a syngas stream (100), in said syngas stage (A), said syngas stream (100) having a higher CO content than the first feed (1) and the second feed (2), or the combined feed (8);
feeding said syngas stream (100) from the syngas stage (A) to the synthesis stage (B);
producing a product stream (200) and an off-gas stream (300) from said syngas stream in said synthesis stage (B);
supplying at least a portion of the off-gas stream (300, 320) from the synthesis stage (B) to said nitrogen removal stage (C) and separating it into a nitrogen-rich purge stream (350) and a purified off-gas stream (400); and
supplying at least a portion of the purified off-gas stream (400) from the nitrogen removal stage (C) to said syngas stage (A) and converting it to a syngas stream, preferably in admixture with said first and second feeds (1, 2) or said combined feed (8).

Aspect 37. The method according to aspect 36, wherein said syngas stage (A) comprises an electrically heated reverse water gas shift (e-RWGS) section (I), wherein said plant comprises:
first feed (1) comprising hydrogen to the e-RWGS section (I), and second feed (2) comprising carbon dioxide to the e-RWGS section (I); or
a combined feed (8) comprising hydrogen and carbon dioxide to the e-RWGS section (I);
   wherein said method comprises the steps of:
   converting at least a portion of said first feed (1) and at least a portion of said second feed (2)—or at least a portion of said combined feed (8)—into a syngas stream (100) in said e-RWGS section (I), and feeding a syngas stream to the synthesis stage (B); and,
   supplying at least a portion of the purified off-gas stream (400) from the nitrogen removal stage (C) to said e-RWGS section (I) and converting it to a syngas stream, preferably in admixture with said first and second feeds (1, 2) or said combined feed (8).

Aspect 38. The method according to any one of aspects 36-37 in which the synthesis stage (B) comprises a Fischer-Tropsch (F-T) unit, said method comprising converting the syngas stream into at least a raw hydrocarbon product stream (200) and a hydrocarbon-containing off-gas stream (300) in the form of an F-T tail gas stream in the F-T unit.

Aspect 39. The method according to any one of aspects 36-38, further comprising a tail gas treatment stage (E) located between the synthesis stage (B) and the nitrogen removal stage (C), wherein said method further comprises the step of treating at least a portion of the tail gas stream (300) from the synthesis stage (B) in the tail gas treatment stage (E), so as to provide a treated off-gas stream (320) to the nitrogen removal stage (C).

Aspect 40. The method according to aspect 39, wherein the tail gas treatment stage (E) comprises a hydrogenation section, arranged to hydrogenate at least a portion of the tail gas stream (300) from the synthesis stage (B), and optionally wherein the tail gas treatment stage (E) further comprises a water gas shift (WGS) section and a steam feed, arranged downstream said hydrogenation section, further optionally wherein the tail gas treatment stage (E) further comprises a pre-conversion section arranged downstream the water gas shift (WGS) section, and wherein said method further comprises the step of treating at least a portion of the tail gas stream (300) from the synthesis stage (B) in the hydrogenation section gas treatment stage (E), optionally in the WGS section and further optionally in the pre-conversion section.

Aspect 41. The method according to aspect 40, wherein the tail gas treatment stage (E) comprises-in order-a hydrogenation section, a WGS section, a pre-conversation section and an SMR section, and wherein said method further comprises the step of treating at least a portion of the tail gas stream (300) from the synthesis stage (B) in the hydrogenation section gas treatment stage (E), the WGS section, the pre-conversion section and the SMR section in turn.

The present invention has been described with reference to a number of embodiments and figures. However, the skilled person is able to select and combine various embodiments within the scope of the invention, which is defined by the appended claims. All documents referenced herein are incorporated by reference.

1. A plant, said plant comprising:
a. a syngas stage;
b. a synthesis stage; and
c. a nitrogen removal stage
said plant comprising:
a first feed comprising hydrogen to the syngas stage, and a second feed comprising carbon dioxide to the syngas stage; or
a combined feed comprising hydrogen and carbon dioxide to the syngas stage;
wherein said syngas stage is arranged to convert at least a portion of said first feed and at least a portion of said second feed—or at least a portion of said combined feed—into a syngas stream having a higher CO content than the first feed and the second feed, or the combined feed, and feed a syngas stream to the synthesis stage;
wherein said synthesis stage is arranged to receive a syngas stream from the syngas stage and provide at least a product stream and an off-gas stream;
wherein said nitrogen removal stage is arranged to receive at least a portion of the off-gas stream from the synthesis stage and separate it into a nitrogen-rich purge stream and a purified off-gas stream; and
wherein said syngas stage is arranged to receive at least a portion of the purified off-gas stream from the nitrogen removal stage and convert it to a syngas stream.

2. The plant according to claim 1, wherein said syngas stage comprises an electrically heated reverse water gas shift section, wherein said plant comprises:
first feed comprising hydrogen to the e-RWGS section and second feed comprising carbon dioxide to the e-RWGS section; or
a combined feed comprising hydrogen and carbon dioxide to the e-RWGS section;
wherein said e-RWGS section is arranged to convert at least a portion of said first feed and at least a portion of said second feed—or at least a portion of said combined feed—into a syngas stream, and feed a syngas stream to the synthesis stage; and
wherein said e-RWGS section is arranged to receive at least a portion of the purified off-gas stream from the nitrogen removal stage and convert it to a syngas stream.

3. The plant according to claim 1, wherein said syngas stage comprises an methanation section and an autothermal reforming section; wherein said plant comprises:
a first feed comprising hydrogen to the methanation section, and a second feed comprising carbon dioxide to the methanation section; or
a combined feed comprising hydrogen and carbon dioxide to the methanation section;
an oxygen feed to said ATR section;
wherein said methanation section is arranged to convert at least a portion of said first feed and at least a portion of said second feed—or at least a portion of said combined feed—into a methane-containing stream, and;
wherein said ATR section is arranged to receive at least a portion of the methane-containing stream from the methanation section, and said oxygen feed, and convert them to a fourth syngas stream, and wherein said fourth syngas stream is arranged to be fed to said synthesis stage.

4. The plant according to claim 1, wherein said plant additionally comprises a third feed comprising hydrocarbons to the syngas stage.

5. The plant according to claim 4, wherein the ratio of moles of carbon in the third feed comprising hydrocarbons, when external to the plant, to the moles of carbon in CO₂ in the second feed is less than 0.3.

6. The plant according claim 1, wherein the content of methane in the synthesis gas stream sent to the synthesis stage is less than 5%.

7. The plant according claim 1, further comprising a tail gas treatment stage located between the synthesis stage and the nitrogen removal stage, said tail gas treatment stage being arranged to treat at least a portion of the tail gas stream from the synthesis stage, and provide a treated off-gas stream to the nitrogen removal stage.

8. The plant according to claim 7, wherein the tail gas treatment stage comprises a hydrogenation section, arranged to hydrogenate at least a portion of the tail gas stream from the synthesis stage, and optionally wherein the tail gas treatment stage further comprises a water gas shift section and a steam feed, arranged downstream said hydrogenation section, further optionally wherein the tail gas treatment stage further comprises a pre-conversion section arranged downstream the water gas shift section.

9. The plant according to claim 8, wherein the tail gas treatment stage comprises—in order—a hydrogenation section, a WGS section, a pre-conversation section and an SMR section.

10. The plant according to claim 1, wherein the nitrogen removal stage comprises a first component removal section, wherein said first component removal section is arranged to receive the off-gas stream and separate it into a third component stream being rich in hydrogen and CO₂, and a fourth component stream being rich in nitrogen and hydrocarbons.

11. The plant according to claim 10, wherein the nitrogen removal stage further comprises a second component removal section, wherein said second component removal section is arranged to receive the fourth component stream from the first component removal section and separate it into a fifth component stream being rich in hydrocarbons and a sixth component stream being rich in nitrogen.

12. The plant according to claim 10, wherein the nitrogen removal stage comprises a compressor section, said compressor section being arranged to compress said third component stream, said fifth component stream, or a mixture of said third and said fifth component streams.

13. The plant according to claim 1, wherein the syngas stream at the inlet of said synthesis stage has a hydrogen/carbon monoxide ratio in the range 1.00-4.00.

14. The plant according claim 1, wherein the ratio of H₂:CO₂ provided at the plant inlet is between 1.0-9.0.

15. The plant according to claim 14, wherein the synthesis stage is an FT synthesis stage and the H₂:CO₂-ratio provided at the plant inlet is in the range of 3.0-7.0.

16. The plant according to claim 1, wherein the synthesis stage comprises a Fischer-Tropsch unit arranged to convert said syngas stream into at least a raw hydrocarbon product stream and a hydrocarbon-containing off-gas stream in the form of an F-T tail gas stream.

17. A method for producing a product stream, said method comprising the steps of:
providing a plant as defined in claim 1;
supplying first feed comprising hydrogen to the syngas stage, and a second feed comprising carbon dioxide to the syngas stage; or
supplying a combined feed comprising hydrogen and carbon dioxide to the syngas stage;
optionally, supplying at least a part of a third feed comprising hydrocarbons, to the syngas stage;
converting at least a portion of said first feed and at least a portion of said second feed—or at least a portion of said combined feed—into a syngas stream, in said syngas stage, said syngas stream having a higher CO content than the first feed and the second feed, or the combined feed;
feeding said syngas stream from the syngas stage to the synthesis stage;
producing a product stream and an off-gas stream from said syngas stream in said synthesis stage;
supplying at least a portion of the off-gas stream from the synthesis stage to said nitrogen removal stage and separating it into a nitrogen-rich purge stream and a purified off-gas stream; and
supplying at least a portion of the purified off-gas stream from the nitrogen removal stage to said syngas stage and converting it to a syngas stream.

18. The method according to claim 17, wherein said syngas stage comprises an electrically heated reverse water gas shift section, wherein said plant comprises:
first feed comprising hydrogen to the e-RWGS section, and second feed comprising carbon dioxide to the e-RWGS section; or
a combined feed comprising hydrogen and carbon dioxide to the e-RWGS section
wherein said method comprises the steps of:
converting at least a portion of said first feed and at least a portion of said second feed—or at least a portion of said combined feed—into a syngas stream in said e-RWGS section, and feeding a syngas stream to the synthesis stage; and,
supplying at least a portion of the purified off-gas stream from the nitrogen removal stage to said e-RWGS section and converting it to a syngas stream.

19. The method according to claim 17, in which the synthesis stage comprises a Fischer-Tropsch unit, said method comprising converting the syngas stream into at least a raw hydrocarbon product stream and a hydrocarbon-containing off-gas stream in the form of an F-T tail gas stream in the F-T unit.

20. The method according to claim 17, further comprising a tail gas treatment stage located between the synthesis stage and the nitrogen removal stage, wherein said method further comprises the step of treating at least a portion of the tail gas stream from the synthesis stage in the tail gas treatment stage, so as to provide a treated off-gas stream to the nitrogen removal stage.

21. The method according to claim 20, wherein the tail gas treatment stage comprises a hydrogenation section, arranged to hydrogenate at least a portion of the tail gas stream from the synthesis stage, and optionally wherein the tail gas treatment stage further comprises a water gas shift section and a steam feed, arranged downstream said hydrogenation section, further optionally wherein the tail gas treatment stage further comprises a pre-conversion section arranged downstream the water gas shift section, and wherein said method further comprises the step of treating at least a portion of the tail gas stream from the synthesis stage in the hydrogenation section gas treatment stage, optionally in the WGS section and further optionally in the pre-conversion section.

22. The method according to claim 21, wherein the tail gas treatment stage comprises—in order—a hydrogenation section, a WGS section, a pre-conversation section and an SMR section, and wherein said method further comprises the step of treating at least a portion of the tail gas stream from the synthesis stage in the hydrogenation section gas treatment stage, the WGS section, the pre-conversion section and the SMR section in turn.
